(19) United States
(12) Patent Application Publication
(10) Pub. No.: US 2026/0088136 A1
(43) Pub. Date: Mar. 26, 2026

(54) METHOD IMPLEMENTED IN WASTEWATER MONITORING SYSTEM FOR EVALUATING BIOAVAILABILITY OF ORGANIC NITROGEN IN WASTEWATER

(71) Applicant: Nanjing University, Nanjing (CN)

(72) Inventors: Haidong HU, Nanjing (CN); Huazai CHENG, Nanjing (CN); Kewei LIAO, Nanjing (CN); Jinfeng WANG, Nanjing (CN); Bing WU, Nanjing (CN); Hongqiang REN, Nanjing (CN)

(21) Appl. No.: 19/405,382

(22) Filed: Dec. 1, 2025

Related U.S. Application Data
(63) Continuation-in-part of application No. 17/588,221, filed on Jan. 29, 2022.

(30) Foreign Application Priority Data
Dec. 28, 2021 (CN) 202111627228.X

(51) Int. Cl. G16C 20/20; C02F 1/00; G06N 20/00; G16C 20/70

(52) U.S. Cl. CPC G16C 20/20; C02F 1/008; G06N 20/00; G16C 20/70; C02F 2209/006

(57) ABSTRACT

A method implemented in a wastewater monitoring system for evaluating bioavailability of organic nitrogen in wastewater, including: obtaining, by using a Fourier Transform Ion Cyclotron Resonance Mass Spectrometer, molecular composition information of organic nitrogen in a wastewater sample collected from a wastewater treatment plant; obtaining bioavailability data corresponding to the wastewater sample, where the bioavailability data is measured through algal bio-culture; training, by a processor of the wastewater monitoring system, a random forest model using the molecular composition information and the bioavailability data; receiving, from the spectrometer, molecular composition information of organic nitrogen in wastewater from a target wastewater treatment plant; and executing, by the processor, the trained machine learning model on the received molecular composition information to generate a predicted bioavailability value; and transmitting, by the wastewater monitoring system, the predicted bioavailability value to a process control unit of the wastewater treatment plant for real-time monitoring or process adjustment.

### CROSS-REFERENCE TO RELATED APPLICATIONS

This application is a continuation-in-part of U.S. application Ser. No. 17/588,221 filed Jan. 29, 2022, now pending, and claims the benefit of Chinese Patent Application No. 202111627228.X filed Dec. 28, 2021, the contents of which, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, MA 02142.

### BACKGROUND

The disclosure belongs to the field of wastewater treatment, and more particularly to a method implemented in a wastewater monitoring system for evaluating bioavailability of organic nitrogen in wastewater.

Conventionally, the bioavailability of organic nitrogen in wastewater is measured by algae bioassay. An algae inoculation solution, a sludge mixed solution, and a wastewater sample are mixed and then cultured for 14 to 28 days in an artificial climate chamber, and the bioavailability of organic nitrogen is represented by the percentage of the organic nitrogen consumed during the culture process in the total organic nitrogen. However, this evaluation method has some disadvantages such as long culture time and strict culture condition, and is thus difficultly applied in continuous monitoring of the bioavailability of organic nitrogen in wastewater from wastewater treatment plants.

### SUMMARY

The disclosure provides a method implemented in a wastewater monitoring system for evaluating bioavailability of organic nitrogen in wastewater.

The wastewater monitoring system comprises a first wastewater treatment plant, a second wastewater treatment plant, a first Fourier Transform Ion Cyclotron Resonance Mass Spectrometer, a second Fourier Transform Ion Cyclotron Resonance Mass Spectrometer, a processor, and a process control unit; the first Fourier Transform Ion Cyclotron Resonance Mass Spectrometer is coupled to the first wastewater treatment plant and configured to continuously analyze organic nitrogen composition data; the second Fourier Transform Ion Cyclotron Resonance Mass Spectrometer is coupled to the second wastewater treatment plant; the processor is coupled to the first Fourier Transform Ion Cyclotron Resonance Mass Spectrometer, the second Fourier Transform Ion Cyclotron Resonance Mass Spectrometer, and the process control unit; and the process control unit is coupled to the second wastewater treatment plant and configured to automatically adjust operational parameters of the second wastewater treatment plant based on a bioavailability value to maintain effluent nitrogen concentrations below regulatory thresholds.

The method comprising:
(1) obtaining, by using the first Fourier Transform Ion Cyclotron Resonance Mass Spectrometer, first molecular composition information of organic nitrogen in a plurality of wastewater samples collected from the first wastewater treatment plant; and obtaining bioavailability data corresponding to the first molecular composition information, wherein the bioavailability data is measured through algal bio-culture experiments;
(2) training, by the processor, a random forest model using the first molecular composition information and the bioavailability data to obtain a trained random forest model, wherein the trained random forest model is configured to predict the bioavailability value based on the first molecular composition information and the corresponding bioavailability data;
(3) receiving, by the processor from the second Fourier Transform Ion Cyclotron Resonance Mass Spectrometer, second molecular composition information of organic nitrogen in wastewater from the second wastewater treatment plant; and
(4) inputting, by the processor, the received second molecular composition information into the trained random forest model to generate a predicted bioavailability value; transmitting, by the processor, the predicted bioavailability value to the process control unit; and adjusting, by the process control unit, the operational parameters of the second wastewater treatment plant based on the predicted bioavailability value.

In a class of this embodiment, the method further comprising: adjusting, by the process control unit, the operational parameters selected from the group consisting of aeration rate, hydraulic retention time, sludge retention time, nutrient dosing rate, and recirculation ratio, based on comparison of the predicted bioavailability value to a target bioavailability range.

In a class of this embodiment, in (2), the random forest model is trained by:
(a) extracting molecular descriptors from the first molecular composition information as feature values and performing data standardization on the feature values to obtain standardized feature values;
(b) ranking the standardized feature values according to feature importance derived from a feature importance metric of the random forest model, and removing feature values having importance below a predefined threshold;
(c) dividing the first molecular composition information and corresponding bioavailability data obtained in (1) into a training set, a validation set, and a test set, training the random forest model on the training set, and optimizing model parameters using the validation set; and
(d) training the random forest model using the model parameters optimized in (c), evaluating a performance of the trained random forest model using the test set, and deploying the trained random forest model in the wastewater monitoring system.

In a class of this embodiment, in (a), the molecular descriptors comprise: molecular parameters of all organic nitrogen molecules; and molecular parameters of organic nitrogen molecules classified into seven molecule categories. the molecular parameters of all organic nitrogen molecules comprise: a mass-to-charge ratio m/z of all organic nitrogen molecules, a number C of carbon atoms of all organic nitrogen molecules, a number H of hydrogen atoms of all organic nitrogen molecules, a number O of oxygen atoms of all organic nitrogen molecules, a number N of nitrogen atoms of all organic nitrogen molecules, a ratio O/C of the number of oxygen atoms to the number of carbon atoms, a ratio H/C of the number of hydrogen atoms to the number of carbon atoms, a number DBE of double bond equivalents, a ratio DBE/H of the number of double bond equivalents to the number of hydrogen atoms, a ratio DBE/O of the number of double bond equivalents to the number of oxygen atoms, a ratio (DBE−O)/C of a difference between the number of double bond equivalents and the number of oxygen atoms to the number of carbon atoms, an average value of a nominal oxidation state of carbon (NOSC) of all organic nitrogen molecules, and intensity-weighted average values of molecular parameters, each intensity-weighted average value being equal to a sum of products obtained by multiplying a relative peak strength of each molecule by the corresponding one of m/z, C, H, O, N, O/C, H/C, DBE, DBE/H, DBE/O, (DBE−O)/C and NOSC.

In a class of this embodiment, the seven molecule categories comprise: lipids, proteins/amino sugars, carbohydrates, unsaturated hydrocarbons, lignin, tannins, and condensed aromatics. screening conditions for the seven molecule categories comprise: lipids: O/C<0.2 and 1.7<H/C<2.2; proteins/amino sugars: 0.2<O/C<0.6, 1.5<H/C<2.2 and N/C≥0.05; carbohydrates: 0.6<O/C<1.0 and 1.5<H/C<2.2; unsaturated hydrocarbons: O/C<0.1, 0.7<H/C<1.5; lignin: 0.1<O/C<0.6, 0.6<H/C<1.7, and a modified aromaticity index AImod<0.67; tannins: 0.6<O/C<1.0, 0.5<H/C<1.5 and a modified aromaticity index AImod<0.67; and condensed aromatics: O/C<1.0, 0.3<H/C<0.7 and a modified aromaticity index AImod≥0.67.

In a class of this embodiment, the molecular parameters of organic nitrogen molecules classified into seven molecule categories comprise: a mass-to-charge ratio m/zi of each molecule category, a number DBEi of double bond equivalents of each molecule category, an average nominal oxidation state of carbon NOSCi of organic nitrogen molecules within each molecule category, a proportion Numi of organic nitrogen molecules belonging to each molecular category, and intensity-weighted average values of molecular parameters, each intensity-weighted average value being equal to a sum of products obtained by multiplying a relative peak strength of organic nitrogen molecules by the corresponding one of m/zi, DBEi and NOSCi, wherein i represents the molecule category.

In a class of this embodiment, the data standardization performed in (a) comprises: computing, for each feature value, a standardized feature value z according to the formula:

$$z = \frac{( x - u )}{s} ;$$

where z is a standardized feature value, x is an original feature value, u is an average value of the feature values, and s is a standard deviation of the feature values.

In a class of this embodiment, in (b), ranking the feature values by importance and removing feature values having importance below a predefined threshold comprises: using a recursive feature elimination algorithm with cross-validation, selecting a gradient-boosting-based learning estimator, and using a determination coefficient R² as a scoring basis for cross-validation; and wherein one feature number is removed from a current feature value set in each iteration, the recursive feature elimination algorithm is repeatedly executed on an updated feature value set until the cross-validation score of the model decreases due to the removal, and feature values to be removed are determined based on feature-importance ranking.

In a class of this embodiment, the first molecular composition information and corresponding bioavailability data obtained in 1) are randomly divided into the training set and the test set at a ratio of 9:1, a sample set is constructed by randomly selecting m samples from the training set, k attributes are randomly selected from an attribute set at each node of a base decision tree using a decision tree as a base learner, and one attribute is selected from the k attributes for node splitting; sampling is performed T times to construct T sample sets each containing m training samples, and one decision tree is trained based on each sample set; a random forest model is constructed from the T decision trees, and a final predicted value of the random forest model is expressed as:

$$\check{f} ( x ) = \frac{1}{T} {\sum }_{i = 1}^{T} T ( x ) ;$$

where f̌(x) is the final predicted value of the random forest model, Tis the number of decision trees, and T(x) is an output value of each decision tree; the training set is processed with a 5-fold cross-validation to adjust model parameters and train the random forest model, and the adjusted model parameters are evaluated using the validation set.

In a class of this embodiment, the model parameters to be adjusted and the corresponding parameter ranges comprise: a number of decision trees from 100 to 10000, a maximum depth of the decision trees from 5 to 55, a minimum impurity reduction threshold from 0.0 to 0.1; the model parameters are combined by random sampling to generate parameter combinations; based on the parameter combinations, additional parameter values within a proximity range are selected, and all combinations thereof are evaluated to identify a parameter combination for training the random forest model.

In a class of this embodiment, in (d), the random forest model is trained using the model parameters optimized in (c), the performance of the random forest model is evaluated using the test set; the evaluation is performed using a determination coefficient R² and a root mean square error RMSE as evaluation metrics, wherein the determination coefficient R² is calculated according to the formula:

$${R}^{2} ( y , \check{y} ) = 1 - \frac{{\sum }_{i = 1}^{n} {( {y}_{i} - {\check{y}}_{i} )}^{2}}{{\sum }_{i = 1}^{n} {( {y}_{i} - \overline{y} )}^{2}} ;$$

the root mean square error RMSE is calculated according to the formula:

$$RMSE = \sqrt{\frac{1}{n} {\sum }_{i = 1}^{n} {( {\check{y}}_{i} - {y}_{i} )}^{2} ;}$$

where yᵢ is a measured value, y̌ᵢ is a predicted value,

$$\overline{y} = \frac{1}{n} {\sum }_{i = 1}^{n} {y}_{i} ,$$

and n is a number of wastewater samples.

The following advantages are associated with the disclosed method.

The disclosed method requires only a small number of wastewater samples for evaluating the bioavailability of organic nitrogen in wastewater, and does not require performing an algal cultivation experiment, thereby significantly shortening a testing period. In addition, the predicted bioavailability value of organic nitrogen in wastewater can be generated immediately after the molecular composition information of organic nitrogen is obtained by the Fourier transform ion cyclotron resonance mass spectrometer, and an average prediction accuracy exceeding 90% can be achieved.

(2) The disclosed method is simple to operate, and the bioavailability of organic nitrogen in wastewater can be obtained by inputting the molecular composition information of organic nitrogen in the wastewater sample into the trained random forest model, such that labor-intensive experimental operations, including an algal cultivation experiment, are avoided.

(3) The disclosed method is completed within 4-6 hours, comprising: solid-phase extraction (SPE) enrichment of the wastewater sample for 3-5 hours; acquisition of molecular composition information using the Fourier Transform Ion Cyclotron Resonance Mass Spectrometer for 1 hour; data preprocessing for 1 minute; and execution of the trained random forest model for 1 minute. As a result, the wastewater monitoring system outputs a predicted bioavailability value on the same day as sample collection. The disclosed method enables municipal wastewater treatment plant personnel to rapidly assess changes in effluent DON bioavailability and to promptly adjust operational parameters of the treatment process in response to dynamic water quality variations.

### BRIEF DESCRIPTION OF THE DRAWINGS

FIG. 1 is a graph of a determination of a number of feature values in machine learning according to the disclosure;

FIG. 2 is a schematic diagram of a division of a dataset of for training the random forest model according to the disclosure;

FIG. 3 is a schematic structure diagram of the second decision tree in the random forest model according to the disclosure;

FIG. 4 is a graph of a performance evaluation of the trained random forest model on the validation set and the test set according to the disclosure; and

FIG. 5 is a graph of an output of the trained random forest model based on a SHAP analysis according to the disclosure.

### DETAILED DESCRIPTION

To further illustrate, embodiments detailing a method implemented in a wastewater monitoring system for evaluating bioavailability of organic nitrogen in wastewater are described below. It should be noted that the following embodiments are intended to describe and not to limit the disclosure.

### Example 1

A wastewater monitoring system comprises one or more municipal wastewater treatment plants, a second wastewater treatment plant, one or more first Fourier Transform Ion Cyclotron Resonance Mass Spectrometer, a second Fourier Transform Ion Cyclotron Resonance Mass Spectrometer, a processor, and a process control unit; the one or more Fourier Transform Ion Cyclotron Resonance Mass Spectrometers are respectively coupled to the one or more municipal wastewater treatment plants and configured to continuously analyze organic nitrogen composition data; the second Fourier Transform Ion Cyclotron Resonance Mass Spectrometer is coupled to the second wastewater treatment plant; the processor is coupled to the one or more first Fourier Transform Ion Cyclotron Resonance Mass Spectrometers, the second Fourier Transform Ion Cyclotron Resonance Mass Spectrometer, and the process control unit; and the process control unit is coupled to the second wastewater treatment plant and configured to automatically adjust at least one operational parameter of the second wastewater treatment plant based on the bioavailability value to maintain effluent nitrogen concentrations below regulatory thresholds.

Wastewater samples were collected from the one or more municipal wastewater treatment plants to evaluate the bioavailability of dissolved organic nitrogen (DON). The average characteristics of the collected wastewater samples were as follows: chemical oxygen demand (COD) concentration of 150.1 mg/L, total nitrogen concentration of 16.2 mg/L, organic nitrogen concentration of 3.2 mg/L, and total phosphorus concentration of 1.1 mg/L. The specific evaluation steps were as follows: (1) Molecular composition information of organic nitrogen in the wastewater samples was measured using the one or more first Fourier Transform Ion Cyclotron Resonance Mass Spectrometers (FT-ICR-MS). Corresponding bioavailability data were obtained using an algal cultivation experiment.

(1) A total of 100 sets of molecular composition information and bioavailability data were collected.

(2) For each wastewater sample, a set of molecular descriptors in the organic nitrogen information was calculated and used as feature values for subsequent model training. The molecular descriptors of organic nitrogen used as the feature values comprised: molecular parameters of all organic nitrogen molecules; and organic nitrogen molecular parameters of seven molecule categories.

The specific calculation process was described below.

The molecular parameters of all organic nitrogen molecules comprised: the average mass-to-charge ratio (m/z) of all organic nitrogen molecules, forming a feature vector x₁=(x₁₁; x₁₂; x₁₃; . . . ; x₁ₙ); the average number of carbon atoms (C), forming a feature vector x₂=(x₂₁; x₂₂; x₂₃; . . . ; x₂ₙ); the average number of hydrogen atoms (H), forming a feature vector x₃=(x₃₁; x₃₂; x₃₃; . . . ; x₃ₙ); the average number of oxygen atoms (O), forming a feature vector x₄=(x₄₁; x₄₂; x₄₃; . . . ; x₄ₙ); the average number of nitrogen atoms (N), forming a feature vector x₅=(x₅₁; x₅₂; x₅₃; . . . ; x₅ₙ); the average O/C ratio, forming a feature vector x₆=(x₆₁; x₆₂; x₆₃; . . . ; x₆ₙ); the average H/C ratio, forming a feature vector x₇=(x₇₁; x₇₂; x₇₃; . . . ; x₇ₙ); the average double bond equivalents (DBE), calculated as

$$DBE = \frac{2 C + N + P - H + 2}{2} ,$$

forming a feature vector x₈=(x₈₁; x₈₂; x₈₃; . . . ; x₈ₙ); the average DBE/H ratio, forming a feature vector x₉=(x₉₁; x₉₂; x₉₃; . . . ; x₉ₙ); the average (DBE/O ratio, forming a feature vector x₁₀=(x₁₀₁; x₁₀₂; x₁₀₃; . . . ; x₁₀ₙ); the average (DBE−O)/C ratio, calculated as

$$( \frac{D B E - O}{C} = \frac{( 2 C + N + P - H + 2 ) / 2 - O}{C} ) ,$$

forming a feature vector x₁₁=(x₁₁₁; x₁₁₂; x₁₁₃; . . . ; x₁₁ₙ); the average nominal oxidation state of carbon (NOSC), calculated as

$$( NOSC = 4 - \frac{4 C + H - 2 O - 3 N - 2 S + 5 P}{C} ,$$

forming a feature vector x₁₂=(x₁₂₁; x₁₂₂; x₁₂₃; . . . ; x₁₂ₙ). Additionally, the sum of intensity-weighted average values of m/z (i.e., m/Z_{wa}=Σ(m/z₁×RIᵢ)) was used to obtain a feature vector x₁₃=(x₁₃₁; x₁₃₂; x₁₃₃; . . . ; x₁₃ₙ); the sum of intensity-weighted average values of C (i.e., C_{wa}=Σ(Cᵢ×RIᵢ)) was used to obtain a feature vector x₁₄=(x₁₄₁; x₁₄₂; x₁₄₃; . . . ; x₁₄ₙ); the sum of intensity-weighted average values of H (i.e., H_{wa}=Σ(HᵢX RIᵢ)) was used to obtain a feature vector x₁₅=(x₁₅₁; x₁₅₂; x₁₅₃; . . . ; x₁₅ₙ); the sum of intensity-weighted average values of O (i.e., O_{wa}=Σ(Oᵢ×RIᵢ) was used to obtain a feature vector x₁₆=(x₁₆₁; x₁₆₂; x₁₆₃; . . . ; x₁₆ₙ); the sum of intensity-weighted average values of N (i.e., N_{wa}=Σ(Nᵢ×RIᵢ)) was used to obtain a feature vector x₁₇=(x₁₇₁; x₁₇₂; x₁₇₃; . . . ; x₁₇ₙ); the sum of intensity-weighted average values of O/C (i.e., O/C_{wa}=Σ(O/Cᵢ×RIᵢ)) was used to obtain a feature vector x₁₈=(x₁₈₁; x₁₈₂; x₁₈₃; . . . ; x₁₈ₙ); the sum of intensity-weighted average values of H/C (i.e., H/C_{wa}=Σ(H/Cᵢ×RIᵢ)) was used to obtain a feature vector x₁₉=(x₁₉₁; x₁₉₂; x₁₉₃; . . . ; x₁₉ₙ); the sum of intensity-weighted average values of DBE (i.e., DBE_{wa}=Σ(DBEᵢ×RIᵢ)) was used to obtain a feature vector x₂₀=(x₂₀₁; x₂₀₂; x₂₀₃; . . . ; x₂₀ₙ); the sum of intensity-weighted average values of DBE/H (i.e., DBE/H_{wa}=Σ(DBE/Hᵢ×RIᵢ)) was used to obtain a feature vector x₂₁=(x₂₁₁; x₂₂₂; x₂₃₃; . . . ; x₂₄ₙ); the sum of intensity-weighted average values of DBE/O (i.e., DBE/O_{wa}=Σ(DBE/Oᵢ×RIᵢ)) was used to obtain a feature vector x₂₂=(x₂₂₁; x₂₂₂; x₂₂₃; . . . ; x₂₂ₙ); the sum of intensity-weighted average values of (DBE−O)/C (i.e., (DBE−O)/C_{wa}=Σ((DBE−O)/Cᵢ×RIᵢ)) was used to obtain a feature vector x₂₃=(x₂₃₁; x₂₃₂; x₂₃₃; . . . ; x₂₃ₙ); and, the sum of intensity-weighted average values of NOSC (i.e., NOSC_{wa}=Σ(NOSCᵢ× RIᵢ)) was used to obtain a feature vector x₂₄=(x₂₄₁; x₂₄₂; x₂₄₃; . . . ; x₂₄ₙ).

All organic nitrogen molecules in each wastewater sample were classified into 7 molecule categories. By way of example, for the lipid category, the molecular parameters of all organic nitrogen molecules were calculated as follows: the average m/z was calculated to form a feature vector x₂₅=(x₂₅₁; x₂₅₂; x₂₅₃; . . . ; x₂₅ₙ); the average DBE was calculated to form a feature vector x₂₆=(x₂₆₁; x₂₆₂; x₂₆₃; . . . ; x₂₆ₙ); the average NOSC was calculated to form a feature vector x₂₇=(x₂₇₁; x₂₇₂; x₂₇₃; . . . ; x₂₇ₙ); the sum of intensity-weighted average values of m/z was used to obtain a feature vector x₂₈=(x₂₈₁; x₂₈₂; x₂₈₃; . . . ; x₂₈ₙ); the sum of intensity-weighted average values of DBE was used to obtain a feature vector x₂₉=(x₂₉₁; x₂₉₂; x₂₉₃; . . . ; x₂₉ₙ); the sum of intensity-weighted average values of NOSC was used to obtain a feature vector x₃₀=(x₃₀₁; x₃₀₂; x₃₀₃; . . . ; x₃₀ₙ); and, the ratio Num₁ of the number of molecules of this category in the number of all molecules in this sample was used to obtain a feature vector x₃₁=(x₃₁₁; x₃₁₂; x₃₁₃; . . . ; x₃₁ₙ). The calculation process of other six molecule categories was the same as above and would not be repeated here.

(3) 73 features values obtained for each wastewater sample were merged, and there are totally 100 wastewater samples, so 100 original sample sets (x₁, x₂, x₃, . . . , x₁₀₀) were obtained. Data standardization was performed on the calculated feature values by the following calculation formula:

$$z = \frac{( x - u )}{s} ;$$

where z was the standardized feature value, x was the original feature value, u was the average value of the feature values, and s was the standard deviation of the feature values. The data of the bioavailability of organic nitrogen in the wastewater samples was incorporated into the standardized original sample sets to obtain an original data set D=((x₁, y₁), (x₂, y₂), (x₃, y₃), . . . , (x₁₀₀, y₁₀₀))=(((−0.137, 0.284, 2.077, . . . , −0.692)^{T}, 48.4), ((−0.912, −0.217, 0.910, . . . , −0.532)^{T}, 58.9), ((0.556, 0.240, −0.148, . . . , −0.315)^{T}, 30), . . . , ((0.407, 0.218, 0.028, . . . , −0.393)^{T}, 30)).

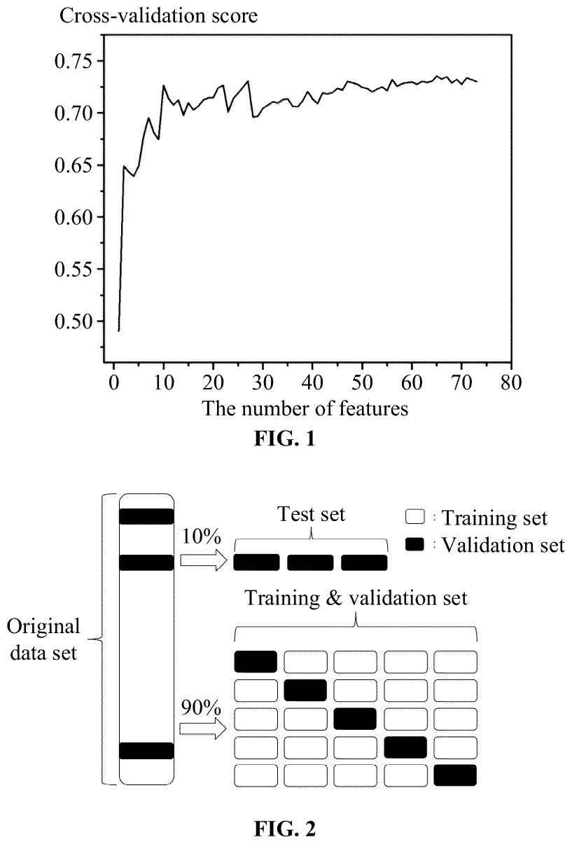

(4) The feature values were ranked by importance, and feature values having importance below a predefined threshold were removed. To perform this process, a recursive feature elimination algorithm with cross-validation was used, and a gradient-boosting-based learning estimator was selected. A determination coefficient R² was employed as the scoring metric for cross-validation. In each iteration, one feature value was removed from the current feature value set, and the recursive feature elimination algorithm was repeatedly executed on the updated feature value set. This process continued until the cross-validation score of the model decreased due to the removal of feature values. The feature values to be removed were determined based on the resulting feature-importance ranking. As illustrated in FIG. 1, the resulting number of selected feature values for training the model was 65, and the specific feature values to be removed were determined according to the feature-importance ranking.

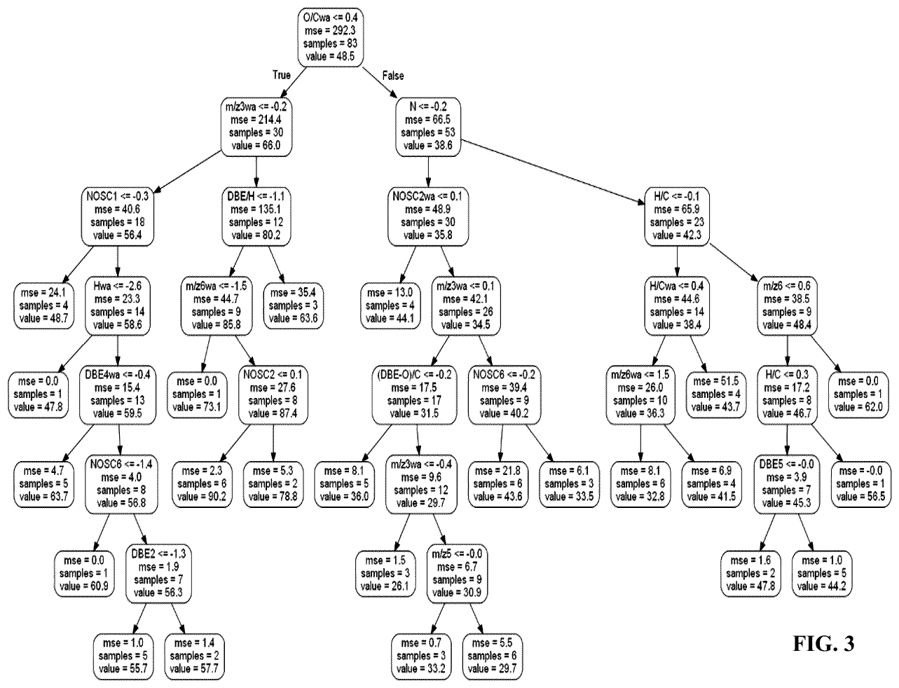

(5) The data set was randomly divided into a training set and a test set at a ratio of 9:1. As shown in FIG. 2, a sample set was constructed by randomly selecting m samples from the training set. At each node of a base decision tree, k attributes were randomly selected from the attribute set, and one attribute was selected from the k attributes for node splitting. Sampling was performed T times to construct T sample sets, each containing mmm training samples. A decision tree was trained based on each sample set, as shown in FIG. 3. A random forest model was constructed from the T decision trees, and the final predicted value of the random forest model was expressed as:

$$\check{f} ( x ) = \frac{1}{T} {\sum }_{i = 1}^{K} T ( x ) ;$$

where f̌(x) was the final predicted value of the random forest model, T was the number of decision trees, and T(x) was the output value of each decision tree. The training set was processed using a 5-fold cross-validation mode to adjust the model parameters and train the random forest model. The adjusted model parameters were evaluated on the validation set. The model parameters to be adjusted and the corresponding ranges thereof were as follows: the number of base decision trees was from 100 to 10000, the maximum depth of the decision tree was from 5 to 55, and the minimum impurity reduction threshold was from 0.0 to 0.1. The model parameters were combined by random sampling to generate the candidate parameter combinations. Based on the parameter combinations, additional parameter values within the parameter proximity range were selected, and all possible combinations of parameters were evaluated to identify the best parameter combination for training the random forest model. The number of final best parameter combinations was equal to the number of base decision trees, i.e., 100; the maximum depth of the decision tree was 15; and, the minimum impurity reduction was 0.05. The random forest model was trained on the training set using the parameter values through 5-fold cross-validation.

(6) The trained random forest model was then evaluated on the test set. The evaluation metrics included a determination coefficient R² and a root mean square error RMSE, where the determination coefficient R² was calculated according to the formula:

$${R}^{2} ( y , \check{y} ) = 1 - \frac{{\sum }_{i = 1}^{n} {( {y}_{i} - {\check{y}}_{i} )}^{2}}{{\sum }_{i = 1}^{n} {( {y}_{i} - \overline{y} )}^{2}} ;$$

where yᵢ was the measured value, y̌ᵢ was the predicted value,

$$\overline{y} = \frac{1}{n} {\sum }_{i = 1}^{n} {y}_{i} ,$$

and n was a number of wastewater samples.

the root mean square error RMSE was calculated according to the formula:

$$R M S E = \sqrt{\frac{1}{n} {\sum }_{i = 1}^{n} {( {\check{y}}_{i} - {y}_{i} )}^{2}} .$$

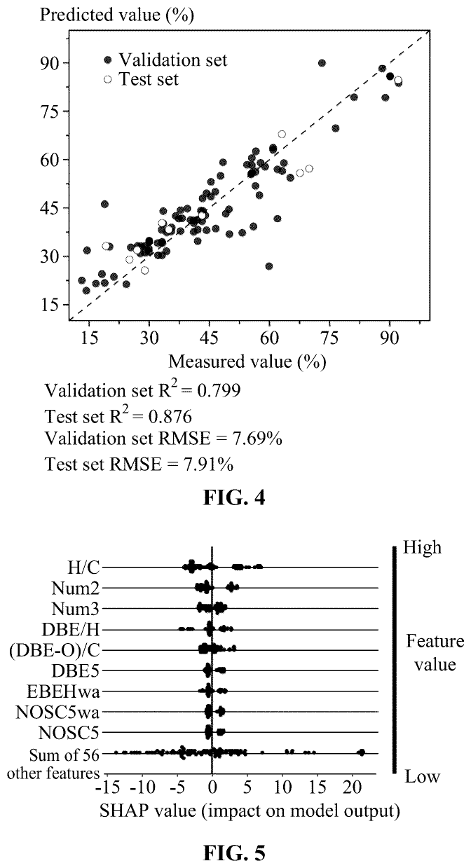

(7) Finally, as shown in FIG. 4, the trained random forest model achieved a determination coefficient R² of 0.779 and a root mean square error (RMSE) of 7.69% on the validation set, and had a determination coefficient R² of 0.879 and a root mean square error (RMSE) of 7.91% on the test set. Furthermore, the predicted bioavailability values of organic nitrogen generated by the random forest model showed no statistically significant difference compared with the bioavailability data measured using the algal cultivation experiment as shown in the following table:

| Difference source | SS | df | MS | F | P-value | F crit |
|---|---|---|---|---|---|---|
| Column | 0.409771 | 1 | 0.409771 | 0.013102 | 0.910931 | 4.844336 |
| Error | 344.0186 | 11 | 31.27442 | | | |
| Total | 344.42837 | 12 | | | | |

The trained random forest model was further interpreted by using a SHAP analysis. As shown in FIG. 5, the analysis had shown that the importance of selected feature values and the influence patterns thereof on the bioavailability of organic nitrogen were consistent with reported findings, indicating that the trained random forest model exhibited robust predictive performance and high reliability.

(8) The molecular composition information of organic nitrogen in printing and dyeing wastewater samples was obtained using the one or more first Fourier Transform Ion Cyclotron Resonance Mass Spectrometers.

(9) The desired feature values were extracted to obtain a feature vector X=(x₁; x₂; x₃; . . . ; x₆₅); and, standardization was performed according to the mean and variance of each feature value in the original dataset to generate a standardized feature vector X=(0.056; −0.138; −0.127; . . . ; −0.323)^{T}.

(10) The standardized feature vector X was input into the trained random forest model, and the random forest model was executed to obtain a predicted bioavailability value of 44.6%. For comparison, the bioavailability of organic nitrogen, measured using algae biological culture was 43.2%. Consistent with the disclosure, there was no significant difference between the predicted bioavailability value of organic nitrogen in wastewater obtained from the trained random forest model and the experimental measurement, resulting in a prediction accuracy of 96.8%.

### Example 2

Wastewater samples from a wastewater plant were selected to evaluate the biodegradability of dissolved organic nitrogen. The average COD concentration of the samples was 35.4 mg/L, the average total nitrogen concentration was 12.8 mg/L, the average organic nitrogen concentration was 0.9 mg/L, and the average total phosphorus concentration was 0.09 mg/L. The specific evaluation steps were described below.

(1) The model was established following the same procedures as described in Example 1.

(2) The molecular composition information of dissolved organic nitrogen in the pharmaceutical wastewater samples was obtained using the one or more Fourier Transform Ion Cyclotron Resonance Mass Spectrometers.

(3) The desired feature values were extracted to construct a feature vector X=(x₁; x₂; x₃; . . . ; x₆₅); and, the feature value was then standardized using the mean and variance of each feature value in the original data set to obtain a standardized feature vector X=(−0.032; −0.284; 2.60; . . . ; −0.571)^{T}.

(4) The standardized feature vector X was input into the trained random forest model, and the prediction model is executed to yield a predicted bioavailability value of 84.6%. For comparison, the bioavailability of organic nitrogen measured via algae biological culture was 92.1%. Consistent with the disclosure, no significant difference was observed between the predicted bioavailability value and the experimental measurement, resulting in a prediction accuracy of 91.9%.

### Example 3

The method for evaluating the biodegradability of dissolved organic nitrogen (DON) in wastewater further comprised the following steps. A wastewater sample obtained from a municipal wastewater treatment plant was used as the test sample.

The dissolved organic nitrogen in the wastewater sample was enriched using a solid-phase extraction (SPE) cartridge. The enrichment process comprised column activation, sample loading, column rinsing, column drying, and column elution. The resulting extract contained an enriched fraction of the dissolved organic nitrogen for subsequent analysis.

The molecular composition information of the dissolved organic nitrogen in the enriched sample was obtained by using the one or more Fourier Transform Ion Cyclotron Resonance Mass Spectrometers (FT-ICR-MS). The instrument was operated with an electrospray ionization (ESI) source in the negative ion mode. The operating parameters included a sample injection rate of 120 μL/h, a capillary voltage of −4.0 kV, an ion accumulation time of 0.06 s, and a mass range from 100 to 1600 Da.

The raw data obtained from FT-ICR-MS were processed using Data Analysis software. Internal calibration was performed using known CHO-type compounds present in dissolved organic matter (DOM). After calibration, molecular formula assignment was carried out under the following conditions: a signal-to-noise ratio (S/N) greater than 3; no limitation on the number of carbon (C), hydrogen (H), and oxygen (O) atoms; and upper limits of 5, 2, and 2 for nitrogen (N), sulfur(S), and phosphorus (P) atoms, respectively. A set of molecular formulas corresponding to the detected peaks was thereby obtained.

The obtained molecular formulas were further screened. The mass error tolerance was set within ±1 ppm. Only molecular formulas satisfying 0.3≤H/C≤2.25 and 0≤O/C≤1 were retained. The double bond equivalent (DBE) value was a non-negative integer, and the number of nitrogen atoms ranged from 1 to 3. When multiple molecular formulas corresponded to the same observed m/z value, the molecular formula having the fewest heteroatoms (N+S+P) was selected. If multiple formulas still remained, the one having the lowest mass error was selected. The analysis was limited to mass spectral peaks within the m/z range of 100-800 Da.

The screened molecular composition formulas were used as input feature values for the trained random forest model employed in the wastewater monitoring system. When the prediction model was executed using the feature values corresponding to the wastewater sample, the model output a predicted bioavailability value of approximately 41.2%. The molecular composition analysis and model prediction were completed within the same day, and the predicted bioavailability value showed no statistically significant difference from a bioavailability value obtained through a conventional algal-cultivation method.

### Comparative Example 1

The example was performed using a conventional algal-cultivation method to evaluate the bioavailability of organic nitrogen in wastewater. In this method, Micractinium pusillum, known as horned green algae cells, in the logarithmic growth phase were first harvested by centrifugation and then inoculated into a nitrogen-free BG11 growth medium. The algal cells were cultured for 1 week until the intracellular nitrogen reserves were depleted, at which point the biomass was considered stable.

The cultivated algae were subsequently inoculated into a wastewater sample collected from a municipal wastewater treatment plant. The inoculation period lasted 14-28 days, after which the change in dissolved organic nitrogen (DON) concentration was measured to obtain the bioavailability data.

Throughout the cultivation period, environmental conditions, including temperature, light intensity, and nutrient levels, must be strictly controlled. Experimental variability among parallel cultures reached 10%, making the conventional method unsuitable for routine or real-time monitoring of DON bioavailability. The entire process was time-consuming, labor-intensive, and required careful operation to ensure reproducibility.

### Comparative Example 2

The example was performed using an enhanced conventional algal-cultivation method to evaluate the bioavailability of organic nitrogen in wastewater. In this method, Selenastrum capricornutum, known as horned green algae cells, in the logarithmic growth phase were first harvested by centrifugation and then inoculated into a nitrogen-free BG11 growth medium. The algal cells were cultured for 1 week until the intracellular nitrogen reserves were depleted, at which point the biomass was considered stable.

The cultivated algae were subsequently inoculated into a wastewater sample collected from a municipal wastewater treatment plant. The inoculation period lasted 14 days, after which the change in dissolved organic nitrogen (DON) concentration was measured to obtain the bioavailability data. DON bioavailability is defined as the ratio of the change in DON concentration to the initial DON concentration. In this example, measurements were repeated three times. The three test results were 55.2%, 62.3%, and 52.1%, respectively. Experimental maximum variability among parallel cultures reached as high as 10%.

According to the method described in Example 3, three portions of the same wastewater sample were subjected to a series of procedures including solid phase, extraction, Fourier Transform Ion Cyclotron Resonance Mass Spectrometry (FT-ICR-MS) analysis, and raw data screening. The model was established following the same procedures as described in Example 1. Referring to Example 2, the standardized feature vectors X for the three replicates were (−1.54; −0.699; 0.979; . . . ; −0.678)^{T}, (−1.12; −0.462; 0.648; . . . ; −0.591)^{T}, (0.670; −0.050; −0.100; . . . ; −0.111)^{T}. These vectors were input into the trained random forest model, and the prediction model was executed to yield predicted bioavailability value of 58.8%, 56.3%, and 57.0%, with a maximum variability of 3% among parallel samples.

Throughout the cultivation period, environmental conditions, including temperature, light intensity, and nutrient levels, must be strictly controlled. Conventional algal-cultivation method requires stringent control of incubation conditions, including temperature, light intensity, and nutrient levels, yet can still yield variability exceeding 10% and takes 14 days to complete. This makes it unsuitable for the real-time, accurate detection of DON bioavailability. In contrast, the present invention achieves results within a single day with a variability margin below 3%.

It will be obvious to those skilled in the art that changes and modifications may be made, and therefore, the aim in the appended claims is to cover all such changes and modifications.

What is claimed is:

1. A method implemented in a wastewater monitoring system for evaluating bioavailability of organic nitrogen in wastewater,
wherein,
the wastewater monitoring system comprises a first wastewater treatment plant, a second wastewater treatment plant, a first Fourier Transform Ion Cyclotron Resonance Mass Spectrometer, a second Fourier Transform Ion Cyclotron Resonance Mass Spectrometer, a processor, and a process control unit; the first Fourier Transform Ion Cyclotron Resonance Mass Spectrometer is coupled to the first wastewater treatment plant and configured to continuously analyze organic nitrogen composition data; the second Fourier Transform Ion Cyclotron Resonance Mass Spectrometer is coupled to the second wastewater treatment plant; the processor is coupled to the first Fourier Transform Ion Cyclotron Resonance Mass Spectrometer, the second Fourier Transform Ion Cyclotron Resonance Mass Spectrometer, and the process control unit; and the process control unit is coupled to the second wastewater treatment plant and configured to automatically adjust operational parameters of the second wastewater treatment plant based on a bioavailability value to maintain effluent nitrogen concentrations below regulatory thresholds;
the method comprising:
(1) obtaining, by using the first Fourier Transform Ion Cyclotron Resonance Mass Spectrometer, first molecular composition information of organic nitrogen in a plurality of wastewater samples collected from the first wastewater treatment plant; and obtaining bioavailability data corresponding to the first molecular composition information, wherein the bioavailability data is measured through algal bio-culture experiments;
(2) training, by the processor, a random forest model using the first molecular composition information and the bioavailability data to obtain a trained random forest model, wherein the trained random forest model is configured to predict the bioavailability value based on the first molecular composition information and the corresponding bioavailability data;
(3) receiving, by the processor from the second Fourier Transform Ion Cyclotron Resonance Mass Spectrometer, second molecular composition information of organic nitrogen in wastewater from the second wastewater treatment plant; and
(4) inputting, by the processor, the received second molecular composition information into the trained random forest model to generate a predicted bioavailability value; transmitting, by the processor, the predicted bioavailability value to the process control unit; and adjusting, by the process control unit, the operational parameters of the second wastewater treatment plant based on the predicted bioavailability value.

2. The method of claim 1, further comprising: adjusting, by the process control unit, the operational parameters selected from the group consisting of aeration rate, hydraulic retention time, sludge retention time, nutrient dosing rate, and recirculation ratio, based on comparison of the predicted bioavailability value to a target bioavailability range.

3. The method of claim 1, wherein in (2), the random forest model is trained by:
(a) extracting molecular descriptors from the first molecular composition information as feature values and performing data standardization on the feature values to obtain standardized feature values;
(b) ranking the standardized feature values according to feature importance derived from a feature importance metric of the random forest model, and removing feature values having importance below a predefined threshold;
(c) dividing the first molecular composition information and corresponding bioavailability data obtained in (1) into a training set, a validation set, and a test set, training the random forest model on the training set, and optimizing model parameters using the validation set; and
(d) training the random forest model using the model parameters optimized in (c), evaluating a performance of the trained random forest model using the test set, and deploying the trained random forest model in the wastewater monitoring system.

4. The method of claim 3, wherein in (a), the molecular descriptors comprise:
molecular parameters of all organic nitrogen molecules; and
molecular parameters of organic nitrogen molecules classified into seven molecule categories;
the molecular parameters of all organic nitrogen molecules comprise: a mass-to-charge ratio m/z of all organic nitrogen molecules, a number C of carbon atoms of all organic nitrogen molecules, a number H of hydrogen atoms of all organic nitrogen molecules, a number O of oxygen atoms of all organic nitrogen molecules, a number N of nitrogen atoms of all organic nitrogen molecules, a ratio O/C of the number of oxygen atoms to the number of carbon atoms, a ratio H/C of the number of hydrogen atoms to the number of carbon atoms, a number DBE of double bond equivalents, a ratio DBE/H of the number of double bond equivalents to the number of hydrogen atoms, a ratio DBE/O of the number of double bond equivalents to the number of oxygen atoms, a ratio (DBE−O)/C of a difference between the number of double bond equivalents and the number of oxygen atoms to the number of carbon atoms, an average value of a nominal oxidation state of carbon (NOSC) of all organic nitrogen molecules, and intensity-weighted average values of molecular parameters, each intensity-weighted average value being equal to a sum of products obtained by multiplying a relative peak strength of each molecule by the corresponding one of m/z, C, H, O, N, O/C, H/C, DBE, DBE/H, DBE/O, (DBE−O)/C and NOSC;
the seven molecule categories comprise: lipids, proteins/amino sugars, carbohydrates, unsaturated hydrocarbons, lignin, tannins, and condensed aromatics;
screening conditions for the seven molecule categories comprise:
$$\text{lipids:} O / C < 0.2 and < 1.7 < H / C < 2.2 ;$$
$$\text{Proteins/amino sugars:} 0.2 < O / C < 0.6 , 1.5 < H / C < 2.2 and N / C \geq 0 \text{.05} ;$$
$$\text{carbohydrates:} 0.6 < O / C < 1. and 1.5 < H / C < 2.2 ;$$
$$\text{unsaturated hydrocarbons:} O / C < 0.1 , 0 \text{.7} < H / C < 1.5 ;$$
$$\text{lignin:} 0.1 < O / C < 0.6 , 0 \text{.6} < H / C < 1.7 , and a modified aromaticity index AImod < 0.67 ;$$
$$\text{tannins:} 0.6 < O / C < 1. , 0 \text{.5} < H / C < 1.5 and a modified aromaticity index AImod < 0.67 ; and$$
$$\text{condensed aromatics:} O / C < 1. , 0.3 < H / C < 0.7 and a modified aromaticity index AImod \geq 0.67 ;$$
and
the molecular parameters of organic nitrogen molecules classified into seven molecule categories comprise: a mass-to-charge ratio m/zi of each molecule category, a number DBEi of double bond equivalents of each molecule category, an average nominal oxidation state of carbon NOSCi of organic nitrogen molecules within each molecule category, a proportion Numi of organic nitrogen molecules belonging to each molecular category, and intensity-weighted average values of molecular parameters, each intensity-weighted average value being equal to a sum of products obtained by multiplying a relative peak strength of organic nitrogen molecules by the corresponding one of m/zi, DBEi and NOSCi, wherein i represents the molecule category.

5. The method of claim 3, wherein the data standardization performed in (a) comprises: computing, for each feature value, a standardized feature value z according to the formula:
$$z = \frac{( x - u )}{s} ;$$
where z is a standardized feature value, x is an original feature value, u is an average value of the feature values, and s is a standard deviation of the feature values.

6. The method of claim 3, wherein in (b), ranking the feature values by importance and removing feature values having importance below a predefined threshold comprises: using a recursive feature elimination algorithm with cross-validation, selecting a gradient-boosting-based learning estimator, and using a determination coefficient R² as a scoring basis for cross-validation; and wherein one feature number is removed from a current feature value set in each iteration, the recursive feature elimination algorithm is repeatedly executed on an updated feature value set until the cross-validation score of the model decreases due to the removal, and feature values to be removed are determined based on feature-importance ranking.

7. The method of claim 3, wherein in (c), the first molecular composition information and corresponding bioavailability data obtained in 1) are randomly divided into the training set and the test set at a ratio of 9:1, a sample set is constructed by randomly selecting m samples from the training set, k attributes are randomly selected from an attribute set at each node of a base decision tree using a decision tree as a base learner, and one attribute is selected from the k attributes for node splitting; sampling is performed T times to construct T sample sets each containing m training samples, and one decision tree is trained based on each sample set; a random forest model is constructed from the T decision trees, and a final predicted value of the random forest model is expressed as:
$$\check{f} ( x ) = \frac{1}{T} {\sum }_{i = 1}^{T} T ( x ) ;$$
where f̌(x) is the final predicted value of the random forest model, Tis the number of decision trees, and T(x) is an output value of each decision tree; the training set is processed with a 5-fold cross-validation to adjust the model parameters and train the random forest model, and the adjusted model parameters are evaluated using the validation set.

8. The method of claim 7, wherein the model parameters to be adjusted and the corresponding parameter ranges comprise: a number of decision trees from 100 to 10000, a maximum depth of the decision trees from 5 to 55, a minimum impurity reduction threshold from 0.0 to 0.1; the model parameters are combined by random sampling to generate parameter combinations; based on the parameter combinations, additional parameter values within a proximity range are selected, and all parameter combinations thereof are evaluated to identify a parameter combination for training the random forest model.

9. The method of claim 3, wherein in (d), the random forest model is trained using the model parameters optimized in (c), the performance of the random forest model is evaluated using the test set; the evaluation is performed using a determination coefficient R² and a root mean square error RMSE as evaluation metrics, wherein the determination coefficient R² is calculated according to the formula:
$${R}^{2} ( y , \check{y} ) = 1 - \frac{{\sum }_{i = 1}^{n} {( {y}_{i} - {\check{y}}_{i} )}^{2}}{{\sum }_{i = 1}^{n} {( {y}_{i} - \overline{y} )}^{2}} ;$$
the root mean square error RMSE is calculated according to the formula:
$$RMSE = \sqrt{\frac{1}{n} {\sum }_{i = 1}^{n} {( {\check{y}}_{i} - {y}_{i} )}^{2}} ;$$
where yᵢ is a measured value, y̌ᵢ is a predicted value,
$$\overline{y} = \frac{1}{n} {\sum }_{i = 1}^{n} {y}_{i} ,$$
and n is a number of wastewater samples.
